(19) United States
(12) Patent Application Publication
(10) Pub. No.: US 2012/0330266 A1
(43) Pub. Date: Dec. 27, 2012

(54) RETICULATED MECHANICAL FASTENING PATCH AND METHOD OF MAKING THE SAME

(75) Inventors: Edwin P. Zonneveld, L'Isle Adam (FR); Thomas J. Gilbert, St. Paul, MN (US); Leigh E. Wood, Woodbury, MN (US); Mark A. Peltier, Forest Lake, MN (US)

(73) Assignee: 3M Innovative Properties Company

(21) Appl. No.: 13/529,517

(22) Filed: Jun. 21, 2012

Related U.S. Application Data
(60) Provisional application No. 61/499,470, filed on Jun. 21, 2011.

(51) Int. Cl. A61F 13/62; A44B 18/00; B23P 13/04

(52) U.S. Cl. 604/391; 29/557; 24/447

(57) ABSTRACT

A reticulated mechanical fastening laminate is disclosed. The reticulated mechanical fastening laminate includes a loop material having a regular pattern of spaced apart geometric shaped openings joined to a carrier. At least the portion of carrier to which the loop material is joined has up to a ten percent elongation. A reticulated mechanical fastening web including the loop material is also disclosed, in which the loop material is not joined to an elastic or pleated extensible carrier. A method of making the mechanical fastener constructions is also disclosed. The method includes providing interrupted slits in a loop material, with each interrupted slit interrupted by at least one intact bridging region of the slit loop material; spreading the slit loop material to provide at least one opening; and fixing of the loop material in a spread configuration.

### CROSS-REFERENCE TO RELATED APPLICATION

This application claims priority to U.S. Provisional Application No. 61/499,470, filed Jun. 21, 2011, the disclosure of which is incorporated by reference in its entirety herein.

### BACKGROUND

Hook and loop fastening systems, where the hook member typically includes a plurality of closely spaced upstanding projections with loop-engaging heads, and the loop member typically includes a plurality of woven, nonwoven, or knitted loops, are useful for providing releasable attachment in numerous applications. For example, hook and loop fastening systems are widely used in wearable disposable absorbent articles to fasten such articles around the body of a person. In typical configurations, a hook strip or patch on a fastening tab attached to the rear waist portion of a diaper or incontinence garment, for example, can fasten to a landing zone of loop material on the front waist region, or the hook strip or patch can fasten to the backsheet (e.g., nonwoven backsheet) of the diaper or incontinence garment in the front waist region. Hook and loop fasteners are also useful for disposable articles such as sanitary napkins. A sanitary napkin typically includes a back sheet that is intended to be placed adjacent to the wearer's undergarment. The back sheet may comprise hook fastener elements to securely attach the sanitary napkin to the undergarment, which mechanically engages with the hook fastener elements.

Some nonwoven materials have been made with openings. Such nonwovens have been attached to elastics or extensible pleated backings. See, e.g., U.S. Pat. Appl. Pub. No. 2004/0147890 (Nakahata et al.), Int. Pat. Appl. Pub. No. WO 1996/10481 (Abuto et al.), and European Patent No. EP 1066008 B1 (Eaton et al.).

### SUMMARY

The present disclosure provides a mechanical fastening laminate or web that comprises loop material with openings. The mechanical fastening laminate or web may include multiple strands of a loop material attached to each other at bridging regions in the loop material and separated from each other between at least some of the bridging regions to provide the openings. The present disclosure also provides an absorbent article that includes the laminate or at least a portion of the web and methods of making the mechanical fastening construction.

In one aspect, the present disclosure provides a method of making a mechanical fastener. The method includes slitting through a loop material to provide a slit loop material having interrupted slits, wherein each interrupted slit is interrupted by at least one intact bridging region of the slit loop material; spreading the slit loop material to provide multiple strands of the loop material attached to each other at least at some of the bridging regions and separated from each other between at least some of the bridging regions to provide at least one opening; and fixing the multiple strands of the loop material in a spread configuration to maintain the at least one opening between the multiple strands.

In another aspect, the present disclosure provides a mechanical fastener made according to the aforementioned method.

In another aspect, the present disclosure provides a reticulated mechanical fastening laminate comprising a loop material having a regular pattern of spaced apart geometric shaped openings joined to a carrier, wherein at least the portion of carrier to which the loop material is joined has up to a ten percent elongation.

In another aspect, the present disclosure provides a reticulated mechanical fastening web comprising a loop material having a regular pattern of spaced apart geometric shaped openings, wherein the loop material is not joined to an elastic or pleated extensible carrier.

The mechanical fastening construction, for example, the reticulated mechanical fastening laminate or web according to and/or made according to the present disclosure, has a unique and attractive appearance, which may be further enhanced by adding a color (e.g., a pigment) to a carrier to which the loop material is attached. Furthermore, the openings can provide breathability and flexibility to the mechanical fastening construction, which may enhance the comfort of the wearer, for example, of an absorbent article comprising the mechanical fastening laminate disclosed herein.

The loop material, for example, in the reticulated mechanical fastening laminate or web according to and/or made according to the present disclosure is able to cover a relatively large area with a relative small amount of material, which may lower the cost of the mechanical fastening laminate or web. The spreading of the loop material may be adjusted based upon, for example, the desired weight or cost in the final product. The methods disclosed herein allow openings to be provided in a loop material to achieve the aforementioned advantages without wasteful material loss. Also, because of the large area that may be covered by the mechanical fastening laminate in an absorbent article, the mechanical fastening laminate may resist shifting forces such as torsional or rotational forces caused by movement of the wearer of the absorbent article.

In this application, terms such as “a”, “an” and “the” are not intended to refer to only a singular entity, but include the general class of which a specific example may be used for illustration. The terms “a”, “an”, and “the” are used interchangeably with the term “at least one”. The phrases “at least one of” and “comprises at least one of” followed by a list refers to any one of the items in the list and any combination of two or more items in the list. All numerical ranges are inclusive of their endpoints and non-integral values between the endpoints unless otherwise stated.

The terms “first” and “second” are used in this disclosure. It will be understood that, unless otherwise noted, those terms are used in their relative sense only. In particular, in some embodiments certain components may be present in interchangeable and/or identical multiples (e.g., pairs). For these components, the designation of “first” and “second” may be applied to the components merely as a matter of convenience in the description of one or more of the embodiments.

When it is said that an interrupted slit “extends” in a particular direction, it is meant that the slit is arranged or aligned in that direction or at least predominantly in that direction. The slit may be linear. As used herein a “linear” slit can be defined by two points in a line in the loop material. The slit may also be substantially linear, which means that the slit can have a slight curvature or slight oscillation. Some oscillation or curvature may result, for example, from the process of slitting a continuous web as would be understood by a person skilled in the art. Any oscillation or curvature is such that the slit generally does not have a portion that crosses over a row of upstanding posts. The slit may also have a wavy or sawtooth pattern with a small amplitude.

A slit that is cut “through” the backing means that the slit cuts through the entire thickness of the backing.

The terms “multiple” and “a plurality” refer to more than one.

The term “machine direction” (MD) as used above and below denotes the direction of a running, continuous web of the loop material during the manufacturing of the mechanical fastener. When a mechanical fastening web is cut into smaller portions from a continuous web, the machine direction corresponds to the length “L” of the loop patch. As used herein, the terms machine direction and longitudinal direction are typically used interchangeably. The term “cross-direction” (CD) as used above and below denotes the direction which is essentially perpendicular to the machine direction. When a mechanical fastening web is cut into smaller portions from a continuous web, the cross direction corresponds to the width “W” of the loop patch.

For some embodiments, slits (e.g., partial slits) are said to penetrate the thickness of the loop material in a certain percent range. The percent penetration may be calculated as depth of the slit divided by the thickness of the backing, with the quotient multiplied by 100.

The term “nonwoven” when referring to a sheet or web means having a structure of individual fibers or threads which are interlaid, but not in an identifiable manner as in a knitted fabric. Nonwoven fabrics or webs can be formed from various processes such as meltblowing processes, spunbonding processes, spunlacing processes, and bonded carded web processes.

The term “elastic” refers to any material that exhibits recovery from stretching or deformation. Likewise, the term “nonelastic” refers to any material that does not exhibit recovery from stretching or deformation.

“Elongation” in terms of percent refers to {(the extended length—the initial length)/the initial length} multiplied by 100.

The term “surface-bonded” when referring to the bonding of fibrous materials means that parts of fiber surfaces of at least portions of fibers are melt-bonded to the second surface of the backing, in such a manner as to substantially preserve the original (pre-bonded) shape of the second surface of the backing, and to substantially preserve at least some portions of the second surface of the backing in an exposed condition, in the surface-bonded area. Quantitatively, surface-bonded fibers may be distinguished from embedded fibers in that at least about 65% of the surface area of the surface-bonded fiber is visible above the second surface of the backing in the bonded portion of the fiber. Inspection from more than one angle may be necessary to visualize the entirety of the surface area of the fiber.

The term “loft-retaining bond” when referring to the bonding of fibrous materials means a bonded fibrous material comprises a loft that is at least 80% of the loft exhibited by the material prior to, or in the absence of, the bonding process. The loft of a fibrous material as used herein is the ratio of the total volume occupied by the web (including fibers as well as interstitial spaces of the material that are not occupied by fibers) to the volume occupied by the material of the fibers alone. If only a portion of a fibrous web has the second surface of the backing bonded thereto, the retained loft can be easily ascertained by comparing the loft of the fibrous web in the bonded area to that of the web in an unbonded area. It may be convenient in some circumstances to compare the loft of the bonded web to that of a sample of the same web before being bonded, for example, if the entirety of fibrous web has the second surface of the backing bonded thereto.

The above summary of the present disclosure is not intended to describe each disclosed embodiment or every implementation of the present disclosure. The description that follows more particularly exemplifies illustrative embodiments. It is to be understood, therefore, that the drawings and following description are for illustration purposes only and should not be read in a manner that would unduly limit the scope of this disclosure.

### BRIEF DESCRIPTION OF THE DRAWINGS

The disclosure may be more completely understood in consideration of the following detailed description of various embodiments of the disclosure in connection with the accompanying drawings, in which:

FIG. 1A is a schematic top view of an exemplary loop material having interrupted slits, useful for the methods of making a mechanical fastener disclosed herein;

FIG. 1B is a schematic top view of the loop material of FIG. 1A after it is spread to provide openings;

FIG. 1C is a schematic top view of the loop material of FIG. 1A after it is spread to a greater extent than in FIG. 1B;

FIG. 2A is a schematic top view of another exemplary loop material having interrupted slits, useful for the methods of making a mechanical fastening strip disclosed herein;

FIG. 2B is a partial, expanded cross-sectional side view taken along line 2BCD-2BCD of FIG. 2A for some embodiments of the methods according to the present disclosure;

FIG. 2C is a partial, expanded cross-sectional side view taken along line 2BCD-2BCD of FIG. 2A for other embodiments of the methods according to the present disclosure;

FIG. 2D is a schematic top view of the slit loop material of FIG. 2A after it is spread to provide openings;

FIG. 3A is a schematic top view of another exemplary loop material with interrupted slits, useful for the methods of making a mechanical fastening strip disclosed herein;

FIG. 3B is a schematic top view of the slit loop material of FIG. 3A after it is spread to provide openings;

FIG. 4A is a schematic top view of an exemplary loop material having interrupted slits, useful for the methods of making a mechanical fastening disclosed herein;

FIG. 4B is a schematic top view of the slit loop material of FIG. 4A after it is spread to provide openings;

FIG. 4C is a schematic top view of the slit loop material of FIG. 4A after it is spread to a greater extent than in FIG. 4B;

FIG. 5A is a schematic top view of an exemplary fastening laminate according to the present disclosure;

FIG. 5B is a schematic top view of another exemplary fastening laminate according to the present disclosure;

FIG. 6A is a perspective view of an exemplary absorbent article incorporating a mechanical fastener according to and/or made according to the present disclosure; and

FIG. 6B is an expanded view of the loop patch 72 shown in FIG. 6A.

### DETAILED DESCRIPTION

Reference will now be made in detail to embodiments of the disclosure, one or more examples of which are illustrated in the drawings. Features illustrated or described as part of one embodiment can be used with other embodiments to yield still a third embodiment. It is intended that the present disclosure include these and other modifications and variations.

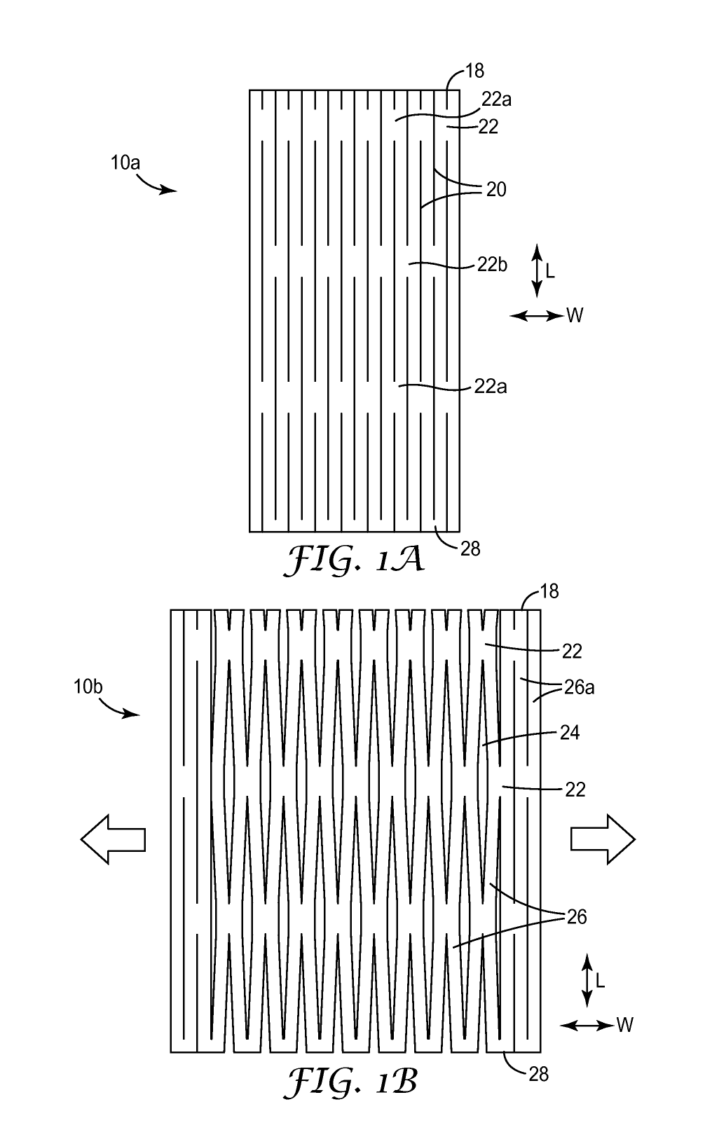

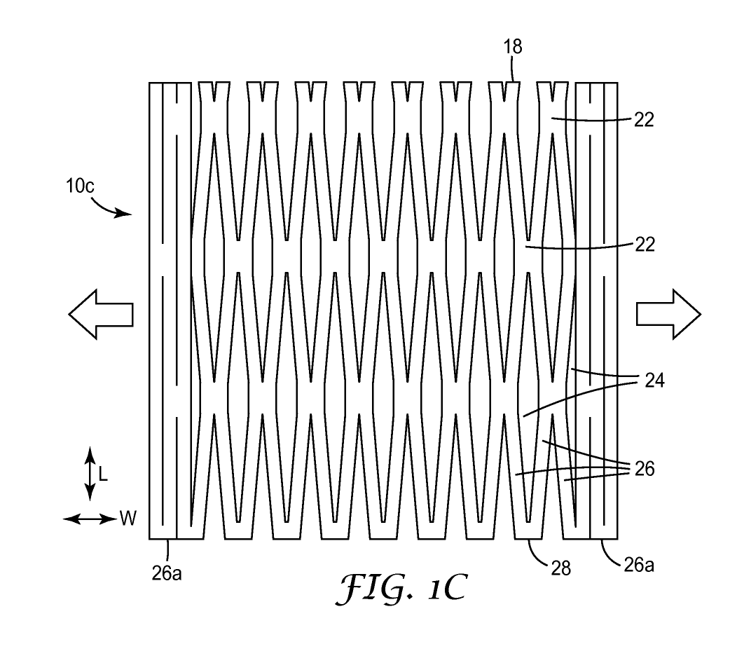

FIG. 1A illustrates an exemplary slit loop material 10a having interrupted slits 20 through the backing, useful for the methods of making a mechanical fastener according to some embodiments of the present disclosure. The illustrated interrupted slits 20 are linear in the direction “L” and extend from the top edge 18 to the bottom edge 28. The interrupted slits are interrupted by intact bridging regions 22 of the loop material 10a. The bridging regions 22 are regions where the loop material is not cut through, and at least a portion of the bridging regions can be considered collinear with interrupted slit 20. In the illustrated embodiment, the interrupted slits 20 are evenly spaced across the width “W” of the strip of loop material although this is not a requirement. Further, in the illustrated embodiment, the bridging regions 22 are staggered in a direction “W” perpendicular to the direction “L” of the interrupted slits 20. The bridging regions 22a and 22b are staggered such that bridging region 22b is located substantially midway between bridging regions 22a in the direction “L”.

FIGS. 1B and 1C illustrate the effect of spreading the slit loop material 10a shown in FIG. 1A to different extents and also illustrate a reticulated mechanical fastening web portion 10b, 10c according to the present disclosure. When the slit loop material 10a is spread in the direction of the arrows shown, multiple strands 26 of the loop material are provided, and the separation between at least some of the multiple strands creates openings 24. Spreading can be carried out to increase the width W of the slit loop material (that is, the dimension in the direction of the spreading) to any extent desired. Increasing the width W of the slit loop material at least 5 percent may be sufficient to provide openings between the multiple strands. In some embodiments, the width W of the slit loop material is increased at least 10, 15, 20, 25, 30, 40, 50 percent. In some embodiments, the width W of the slit loop material is increased up to 70, 100, 200, 250, or 300 percent.

Spreading may be carried out to provide openings between all of the multiple strands 26, or spreading may be carried out so that not all of the multiple strands are spread between the bridging regions 22. In FIGS. 1B and 1C, at least two strands 26a on each edge of the mechanical fastener are not separated. This may be advantageous in some embodiments, for example, to provide a reticulated mechanical fastening strip with a straight edge.

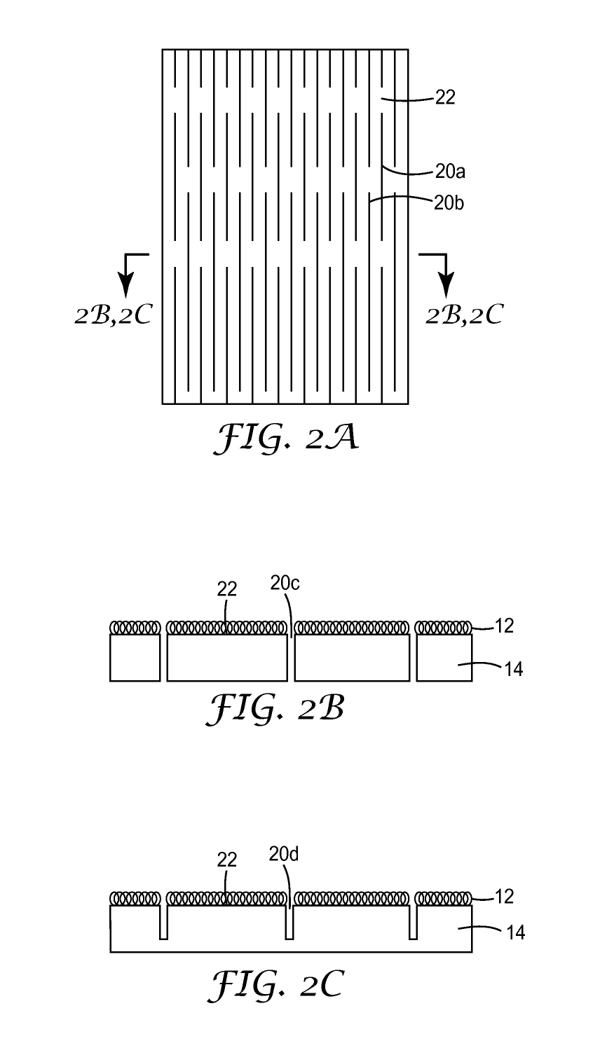

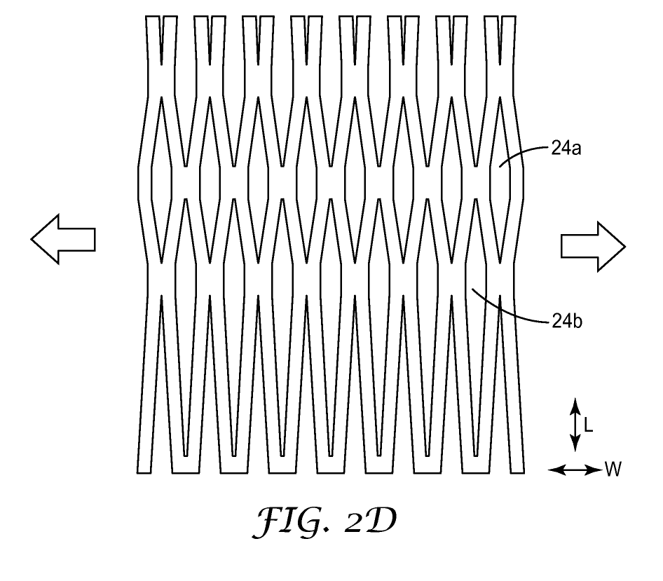

FIG. 2A illustrates an exemplary slit loop material similar to slit loop material 10a shown in FIG. 1A. However, in the embodiment shown in FIG. 2A, slit portions 20a have different lengths than slit portions 20b, which results in openings 24a and 24b having different sizes after the slit loop material is spread as shown in FIG. 2D. The slit portions of the smaller size 20a and slit portions of the larger size 20b each may be aligned with each other across the loop material as shown in FIG. 2A. Or in other embodiments, slits of different sizes may be arranged randomly in the loop material or slits of the same size may be offset relative to each other in a regular pattern.

A partial, expanded view of an exemplary cross-section taken through the slit loop material of FIG. 2A at line 2B, 2C-2B, 2C, which extends through some interrupted slits and some bridging regions, is shown in FIG. 2B. In the illustrated embodiment, the loop material comprises a fibrous layer 12 and a backing 14. The interrupted slits 20c cut through the entire thickness of the loop material. The interrupted slits 20c are made without removing material but are shown out of scale FIG. 2B to make them more easily visible. In other words, the multiple strands of the fibrous layer 12 and backing 14 on either side of the interrupted slits 20c are abutting and not spaced apart. The bridging regions 22 of the loop material are not slit.

The slit loop material shown in FIG. 2A can also be made with partial slits as shown in FIG. 2C. In embodiments of FIG. 2A shown in FIG. 2C, partial slits 20d are cut into the fibrous layer 12 and the backing 14. In the illustrated embodiment, the partial slits 20d are interrupted by bridging regions 22 of the loop material that are not slit. The partial depth slits penetrate the thickness of the loop material to an extent that allows it to open during the spreading shown in FIG. 2D. This penetration may be, for example, at least 60, 65, 70, 75, or 80 percent of the thickness of the loop material and may be, for example, up to 99, 98, 96, or 95 percent of the thickness of the loop material. For example, the penetration may be in a range from 60 to 95, 60 to 90, 65 to 95, 70 to 90, or 65 to 85 percent of the thickness of the loop material. Again, in this embodiment, the partial slits 20d are typically made without removing material from the fibrous layer 12 or the backing 14 but are shown out of scale in FIG. 2C to make them more easily visible. Also, like in FIG. 2B, the bridging regions 22 are not slit.

In the reticulated mechanical fastening strip shown in FIG. 2D, openings 24a and 24b have different sizes. That is, openings 24a are shorter in the longitudinal direction L than openings 24b. It is also possible to make openings that have different widths in a direction W perpendicular to the interrupted slits by using slits of varying lengths. Furthermore, referring again to FIG. 2A, the length of the bridging regions 22 may be made to vary within a strand 26 or between strands 26 as desired for a particular application or appearance.

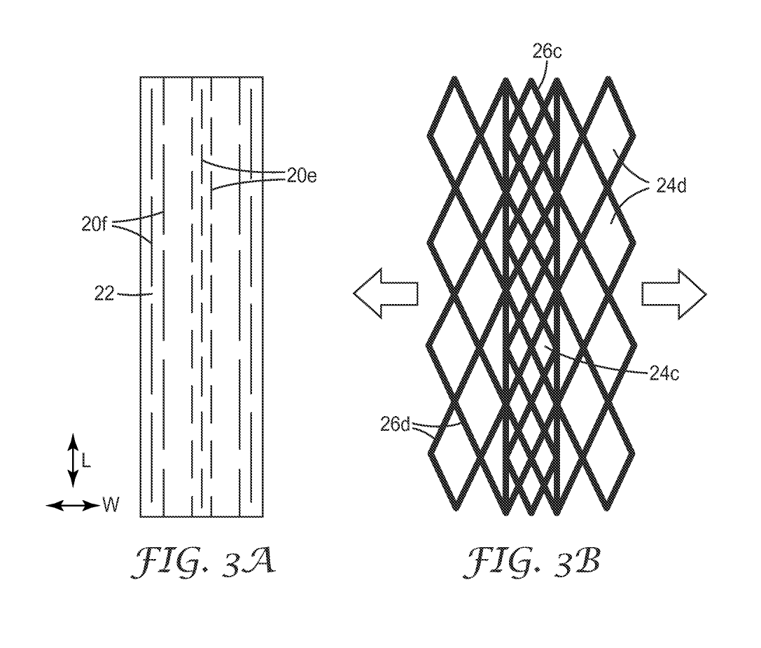

FIG. 3A illustrates an exemplary slit loop material similar to slit loop material 10a shown in FIG. 1A. However, in the embodiment shown in FIG. 3A, slit portions 20e have different lengths than slit portions 20f, which results in openings 24c and 24d having different sizes after the slit loop material is spread as shown in FIG. 3B. In contrast to the embodiment shown in FIGS. 2A-2D, which illustrates interrupted slits with slit portions of different lengths in the longitudinal direction L, and the corresponding resulting openings, FIGS. 3A and 3B illustrate patterns of slit portions of different lengths in the width direction W. The multiple strands 26c and 26d have a different appearance from each other in the same reticulated mechanical fastening strip, for example, multiple strands 26c and 26d zig-zag or undulate with a different wavelength and amplitude.

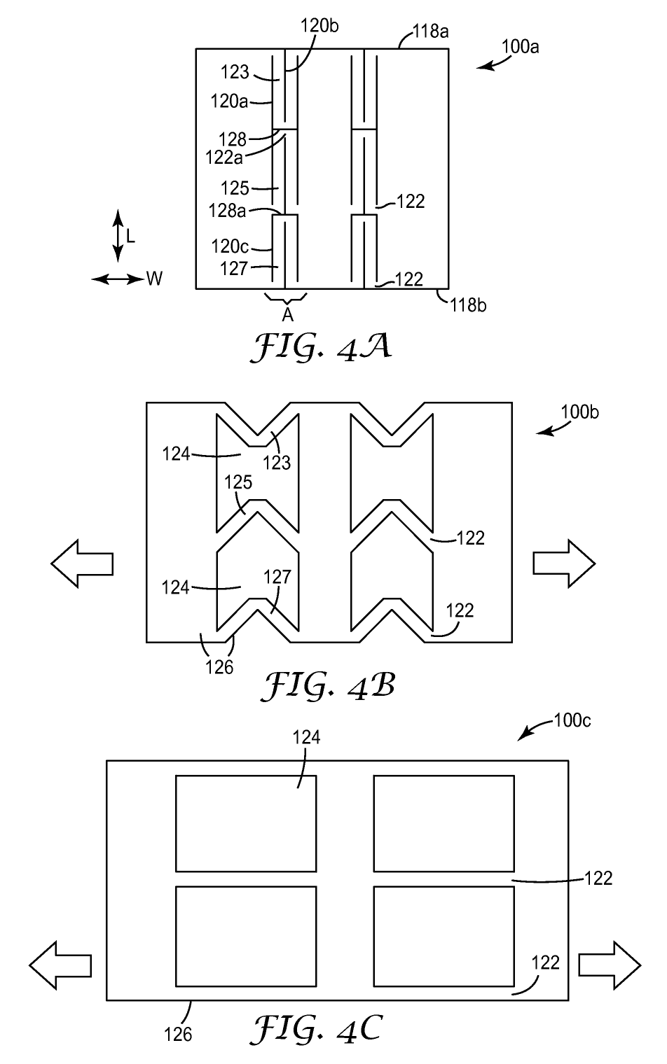

FIGS. 4A-4C illustrate another exemplary method for making a mechanical fastening strip and a resulting reticulated mechanical fastening strip according to the present disclosure. In FIG. 4A, slit loop material 100a is provided with interrupted slits 120a, 120b, and 120c. In the illustrated embodiment, a group of three interrupted slits “A” are positioned together to provide connection regions 123, 125, and 127 when the slit loop material is spread. Each group “A” of three interrupted slits includes a center interrupted slit 120b, which extends through the top and bottom edges 118a and 118b of the loop material 100a. On either side of the center interrupted slit 120b are interrupted slits that do not extend through the top or bottom edges 118a and 118b but include a long slit portion 120a and a shorter slit portion 120c. The slit portions of center interrupted slit 120b are relatively shorter than the long slit portion 120a. At least some of the bridging regions 122a of the center interrupted slit 120b are provided with a transverse slit 128, which is transverse to the direction of interrupted slit 120b. In the illustrated embodiment, transverse slit 128 connects long slit portions 120a on either side of the center interrupted slit 120b. Similarly, transverse slit 128a connects the ends of shorter slit portions 120c on either side of center slit 120b. The result of the arrangement of interrupted slit 120b and slit portions 120a and 120c and transverse slits 128 and 128a is the formation of three connection members 123, 125, and 127 surrounding center interrupted slit 120b that allow the slit loop material 100a to be spread as shown in FIG. 4B.

FIGS. 4B and 4C illustrate the effect of spreading the slit loop material 100a shown in FIG. 4A to different extents and also illustrate a reticulated mechanical fastening strip 100b, 100c according to the present disclosure. When the slit loop material 100a is spread in the direction of the arrows shown, multiple strands 126 are provided, and the separation between at least some of the multiple strands creates openings 124.

Although the methods of making mechanical fastening strip illustrated in FIGS. 1A-1C, 2A-2D, 3A-3B, and 4A-4C each show interrupted slits extending parallel to the longitudinal direction “L” of the loop material, interrupted slits may be made in any desired direction. For example, interrupted slits may be made at an angle from 1 to 90 degrees to the longitudinal direction of the loop material. When the methods disclosed herein are practiced on a continuous web of loop material, interrupted slits may be made in the machine direction, the cross-direction, or any desired angle in between the machine direction and the cross-direction. In some embodiments, interrupted slits may be made at an angle in a range from 35 to 55 degrees (e.g., 45 degrees) to the longitudinal direction “L” of the mechanical fastening strip.

For the embodiments of reticulated mechanical fastening strips or methods of making them illustrated in FIGS. 1A-1C, 2A-2D, 3A-3B, and 4A-4C, the bridging regions 22 and 122 are staggered in a direction “W” perpendicular to the direction “L” of the interrupted slits 20a-e and 120a-c. For example, referring again to FIG. 1A, the bridging regions 22a and 22b are substantially evenly spaced apart in the direction “L” but are staggered in the direction “W”, perpendicular to the direction “L”. When the bridging regions are staggered in this manner, the number of bridging regions necessary to make the slit loop material handle as an integral unit can be minimized. In other embodiments, the bridging regions 22 and 122 are aligned in a direction “W” perpendicular to the direction of the interrupted slits 20a-e and 120a-c.

The particular arrangement of the bridging regions, whether aligned or staggered in a direction perpendicular to the interrupted slits 20a-e and 120a-c, can be designed, for example, based on the desired length of the slits and the amount of spreading desired for the multiple strands 26, 126. Various lengths of bridging regions 22 and 122 may be useful. In some embodiments, any bridging regions 22 and 122 in a given interrupted slit 20a-g and 120a-c have a combined length in the direction of the interrupted slit of up to 50 (in some embodiments, 40, 30, 25, 20, 15, or 10) percent of the length of the loop material in the first direction. In some embodiments, for maximizing the ability of the slit loop material 10a and 100a to spread, it may be desirable to minimize the combined length of the bridging regions in the direction of the interrupted slit. Minimizing the combined length of the bridging regions 22 and 122 in the direction of the interrupted slit may be accomplished by at least one of minimizing the length of any particular bridging region 22 and 122 or maximizing the distance between bridging regions 22 and 122. In some embodiments, the length of one bridging region in the direction of the interrupted slit is up to 3, 2, or 1.5 mm and at least 0.25, 0.5, or 0.75 mm. In some embodiments, the number of bridging regions along the length of the loop material 10a-c and 100a-c in the direction of the interrupted slit is up to 1.5, 1.25, 1.0, 0.75, 0.60, or 0.5 per cm. The distance between bridging regions 22 and 122 in the direction of the interrupted slit may be, for example, at least 0.75, 1.0, 1.25, 1.5, or 1.75 cm. Furthermore, the length of the interrupted slit portions between bridging regions can be adjusted and may be selected to maximize the distance between bridging regions. In some embodiments, the length of the interrupted slit portion between bridging regions is at least 8 (in some embodiments, at least 10, 12, 14, 15, 16, 17, 18, 19, or 20) mm. Typically, the interrupted slits of the slit loop materials disclosed herein have longer slit regions and shorter bridging regions than perforations that are designed to allow easy separation of two parts of a film.

For the embodiments of reticulated mechanical fastening strips or methods of making them illustrated in FIGS. 1A-1C, 2A-2D, 3A-3B, and 4A-C, the interrupted slits may be evenly spaced or unevenly spaced as desired. For interrupted slits that are evenly spaced, the spacing (e.g., distance in the direction “W”) between the interrupted slits may differ by up to 10, 5, 2.5, or 1 percent.

For any of the embodiments of reticulated mechanical fastening strips and methods of making a mechanical fastening strip disclosed herein, the number of interrupted slits and resulting openings may be adjusted depending on the requirements of the application. In some embodiments, there are up to 10, 9, 8, 7, 6, 5, 4, 3, 2, or 1 interrupted slits per 10 mm across the width of the loop material (i.e., in a direction “W” substantially perpendicular to the first direction or machine direction).

For the embodiments of reticulated mechanical fastening strips illustrated in FIGS. 1B-1C, 2D, 3B, and 4B-C, the openings are in the form of a repeating pattern of geometric shapes. In the illustrated embodiments, the geometric shapes are polygons. The shapes may be quadrilaterals such as diamonds, squares, or rectangles. In some embodiments, curved lines may be used, which can result in crescent shaped openings after spreading. As shown in FIG. 3B, there may be more than one repeating pattern of geometric shaped openings. The openings may be evenly spaced or unevenly spaced as desired. For openings that are evenly spaced, the spacing (e.g., distance in the direction “W”) between the openings may differ by up to 10, 5, 2.5, or 1 percent.

For any of the embodiments of reticulated mechanical fastening strips and methods of making them disclosed herein, the reticulated mechanical fastening strip may be in the form of a roll, from which reticulated mechanical fastening patches are cut in a size appropriate to the desired application. In this application, the reticulated mechanical fastening strip may also be a patch that has been cut to a desired size. Furthermore, in some embodiments, including any of the embodiments described above in connection with FIGS. 1A-C, 2A-D, 3A-B, and 4A-C, the loop material has a top edge 18 and a bottom edge 28 and the interrupted slits 20a-g or 120a-c extend from the top edge 18 to the bottom edge 28. In other embodiments, slits can be made cross-web, from side edge to side edge.

The loop material useful for practicing the present disclosure can be made from any suitable material that interlocks with corresponding hook fastening elements. In some embodiments, the loop fastening elements are typically formed from knitted fabrics, woven fabrics, or non-woven fabrics (e.g., spunbond webs, spunlaced webs, airlaid webs, meltblown web, and bonded carded webs). For example, the mechanical fastening patches may include fiber loops projecting from a knitted, woven, or non-woven backing or may be extrusion-bonded, adhesive-bonded, and/or sonically-bonded fiber loops. Useful loop materials may be made of natural fibers (e.g., wood or cotton fibers), synthetic fibers (e.g., thermoplastic fibers), or a combination of natural and synthetic fibers. Exemplary materials for forming thermoplastic fibers include polyolefins (e.g., polyethylene, polypropylene, polybutylene, ethylene copolymers, propylene copolymers, butylene copolymers, and copolymers and blends of these polymers), polyesters, and polyamides. The fibers may also be multi-component fibers, for example, having a core of one thermoplastic material and a sheath of another thermoplastic material.

In some embodiments, the loop material comprises a fibrous layer disposed on a backing. Suitable backings include textiles, paper, thermoplastic films (e.g., single- or multilayered films, coextruded films, laterally laminated films, or films comprising foam layers), and combinations thereof. The thickness of the backing may be up to about 400, 250, 150, 100, 75 or 50 micrometers, depending on the desired application. In some embodiments, the thickness of the backing is in a range from 30 to about 225 micrometers, from about 50 to about 200 micrometers, or from about 100 to about 150 micrometers.

In some embodiments, the backing is a thermoplastic backing. Suitable thermoplastic materials include polyolefin homopolymers such as polyethylene and polypropylene, copolymers of ethylene, propylene and/or butylene; copolymers containing ethylene such as ethylene vinyl acetate and ethylene acrylic acid; polyesters such as poly(ethylene terephthalate), polyethylene butyrate and polyethylene napthalate; polyamides such as poly(hexamethylene adipamide); polyurethanes; polycarbonates; poly(vinyl alcohol); ketones such as polyetheretherketone; polyphenylene sulfide; and mixtures thereof. Typically, the thermoplastic is a polyolefin (e.g., polyethylene, polypropylene, polybutylene, ethylene copolymers, propylene copolymers, butylene copolymers, and copolymers and blends of these materials). In some embodiments, the thermoplastic backing has stretch-induced molecular orientation. In other embodiments, the thermoplastic backing is not provided with macroscopic stretch-induced molecular orientation in the direction of the interrupted slits or in the direction of spreading. In these embodiments, there may be some stress-induced orientation localized in the bridging regions.

Exemplary suitable loop materials are described, for example, in U.S. Pat. Nos. 5,256,231 (Gorman et al.) and 5,389,416 (Mody et al.), the disclosures of which are incorporated herein by reference in their entirety. As described in U.S. Pat. No. 5,256,231 (Gorman et al.), the fibrous layer in a loop material according to some embodiments disclosed herein comprises arcuate portions projecting in the same direction from spaced anchor portions on the backing.

Suitable commercially available mechanical loop materials include knitted and extrusion-bonded loop materials from 3M Company, St. Paul, Minn.

The bridging regions 22 interrupting the interrupted slits 20a allow the slit and/or spread loop material to be handled as an integral unit, for example, to be handled in roll form and converted as desired. Accordingly, in some embodiments, the multiple strands 26 and 126 are not joined to a carrier, at least when the reticulated mechanical fastening strip is initially formed. When the multiple strands are not joined to a carrier, it may mean that the strands are not laminated (e.g., extrusion laminated), adhered, bonded (e.g., ultrasonic bonded or compression bonded) or otherwise attached to a carrier (e.g., a substrate, fastening tab, fastening tape, etc.). Since, in some embodiments, the reticulated mechanical fastening strip according to the present disclosure may be made without being joined to a carrier, there is great flexibility in how the loop material may be converted and subsequently attached to an article to be fastened.

On the other hand, the reticulated mechanical fastening strip according to the present disclosure may be useful in a fastening laminate. The fastening laminate may be a fastening tab or landing zone comprising the reticulated mechanical fastening strip disclosed herein in any of the aforementioned embodiments, or the fastening laminate may comprise a reticulated mechanical fastening strip joined to the backsheet of an absorbent article. In some embodiments, the fastening laminate is useful for joining the front waist region and the rear waist region of an absorbent article. The fastening laminate may comprise a carrier and a reticulated mechanical fastening strip disclosed herein, wherein the loop material is joined to the carrier.

In some embodiments, fixing the multiple strands of the loop material in a spread configuration to maintain the at least one opening between the multiple strands of the loop material comprises joining the multiple strands to a carrier. The multiple strands or reticulated mechanical fastening strip may be joined to a carrier, for example, by lamination (e.g., extrusion lamination), adhesives (e.g., pressure sensitive adhesives), or other bonding methods (e.g., ultrasonic bonding, compression bonding, or surface bonding).

The carrier may be continuous (i.e., without any through-penetrating holes) or discontinuous (e.g. comprising through-penetrating perforations or pores). The carrier may comprise a variety of suitable materials including woven webs, non-woven webs (e.g., spunbond webs, spunlaced webs, airlaid webs, meltblown web, and bonded carded webs), textiles, plastic films (e.g., single- or multilayered films, coextruded films, laterally laminated films, or films comprising foam layers), and combinations thereof. In some embodiments, the carrier is a fibrous material (e.g., a woven, nonwoven, or knit material). In some embodiments, the carrier comprises multiple layers of nonwoven materials with, for example, at least one layer of a meltblown nonwoven and at least one layer of a spunbonded nonwoven, or any other suitable combination of nonwoven materials. For example, the carrier may be a spunbond-meltbond-spunbond, spunbond-spunbond, or spunbond-spunbond-spunbond multilayer material. Or, the carrier may be a composite web comprising a nonwoven layer and a dense film layer.

Fibrous materials that provide useful carriers may be made of natural fibers (e.g., wood or cotton fibers), synthetic fibers (e.g., thermoplastic fibers), or a combination of natural and synthetic fibers. Exemplary materials for forming thermoplastic fibers include polyolefins (e.g., polyethylene, polypropylene, polybutylene, ethylene copolymers, propylene copolymers, butylene copolymers, and copolymers and blends of these polymers), polyesters, and polyamides. The fibers may also be multi-component fibers, for example, having a core of one thermoplastic material and a sheath of another thermoplastic material.

Useful carriers may have any suitable basis weight or thickness that is desired for a particular application. For a fibrous carrier, the basis weight may range, e.g., from at least about 5, 8, 10, 20, 30, or 40 grams per square meter, up to about 400, 200, or 100 grams per square meter. The carrier may be up to about 5 mm, about 2 mm, or about 1 mm in thickness and/or at least about 0.1, about 0.2, or about 0.5 mm in thickness.

One or more zones of the carrier may comprise one or more elastically extensible materials extending in at least one direction when a force is applied and returning to approximately their original dimension after the force is removed. However, in some embodiments, at least the portion of the carrier joined to the multiple strands of the loop material is not stretchable. In some embodiments, the portion of carrier joined to the loop material will have up to a 10 (in some embodiments, up to 9, 8, 7, 6, or 5) percent elongation.

In some embodiments, the carrier may be extensible but nonelastic. In other words, the carrier may have an elongation of at least 5, 10, 15, 20, 25, 30, 40, or 50 percent but substantially no recovery from the elongation (e.g., up to 10 or 5 percent recovery). In embodiments of the methods disclosed herein, wherein the carrier is extensible but nonelastic, spreading the slit loop material to provide multiple strands of the loop material may be carried out after the loop material is joined to the extensible carrier. In these embodiments, fixing the multiple strands of the loop material in a spread configuration to maintain the at least one opening between the multiple strands may be carried out simultaneously with spreading the slit loop material, and fixing the multiple strands may be accomplished by the carrier maintaining its elongation. The multiple strands on the extensible carrier may further be annealed as described in more detail below. Suitable extensible carriers may include nonwovens (e.g., spunbond, spunbond meltblown spunbond, or carded nonwovens). In some embodiments, the nonwoven may be a high elongation carded nonwoven (e.g., HEC). In some embodiments, the carrier is not pleated.

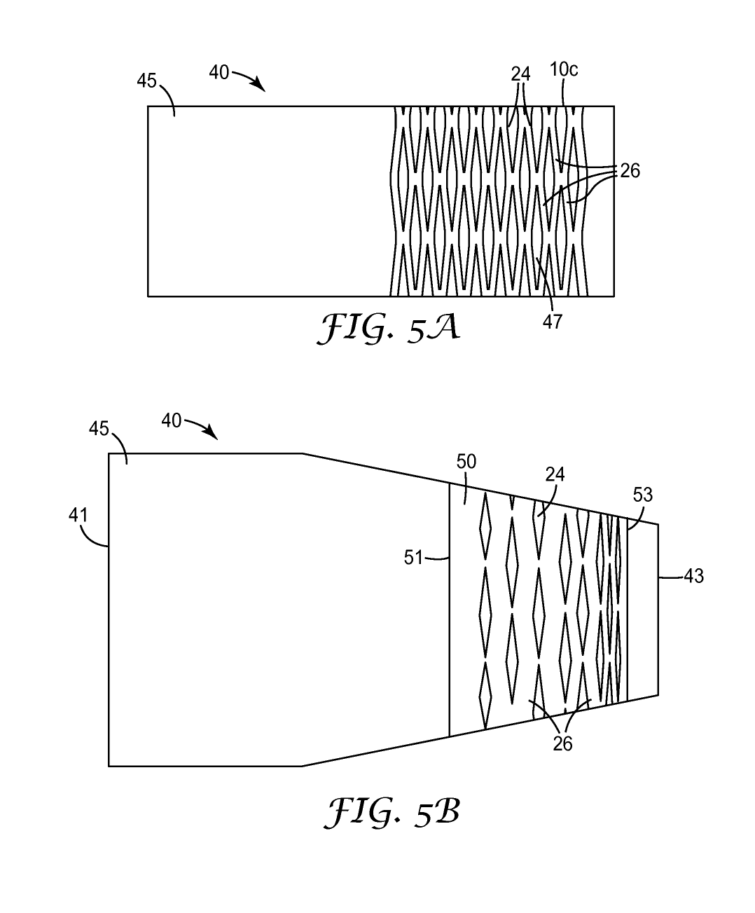

An embodiment of a fastening laminate 40 according to the present disclosure is illustrated in FIG. 5A. Fastening laminate 40 comprises carrier 45 and reticulated mechanical fastening strip 10c, as shown and described in FIG. 1C above. The reticulated mechanical fastening strip includes multiple strands 26 and openings 24 between the strands. Optionally, the fastening laminate 40 can include adhesive 47 between at least a portion of the reticulated mechanical fastening strip and at least a portion of the carrier. In some of these embodiments, there can be exposed adhesive between the multiple strands 26 of the reticulated mechanical fastening strip 10c, which may be advantageous, for example, for allowing the fastening laminate 40 to attach to a surface through a combination of adhesive bonding and mechanical fastening.

Another fastening laminate 40 according to the present disclosure, comprising carrier 45 and reticulated mechanical fastening strip 10c, is illustrated in FIG. 5B. Fastening laminate 40 may be a shaped landing zone (e.g., on an absorbent article) with first edge 41 and an opposing second edge 43. In the embodiment illustrated in FIG. 5B, the carrier 45 is shaped such that the second edge 43 is narrower in the longitudinal direction “L” than the first edge 41. The shape of reticulated mechanical fastening strip 50 corresponds to the shape of the carrier 45 with a second edge 53 narrower in the longitudinal direction “L” than a first edge 51. In the illustrated embodiment, the width of the multiple strands 26 in reticulated mechanical fastening strip 50 varies, and, therefore, the spacing between openings 24 varies. In fastening laminate 40 shown in FIG. 5B, the strands 26 are thinner toward second edge 53 and larger toward first edge 51.

The fastening laminates disclosed herein are useful, for example, in absorbent articles. In some embodiments, absorbent articles according to the present disclosure have at least a front waist region, a rear waist region, and a longitudinal center line bisecting the front waist region and the rear waist region, wherein at least one of the front waist region or the rear waist region comprises the fastening laminate disclosed herein. The fastening laminate may be in the form of a fastening tab or landing zone that is bonded to at least one of the front waist region or the rear waist region. In other embodiments, the fastening laminate may be an integral ear portion of the absorbent article. In these embodiments, the direction of the slits that provide openings (in some embodiments, the machine direction) of the reticulated mechanical fastening strip may generally aligned with the longitudinal center line of the absorbent article or may be transverse to the longitudinal center line.

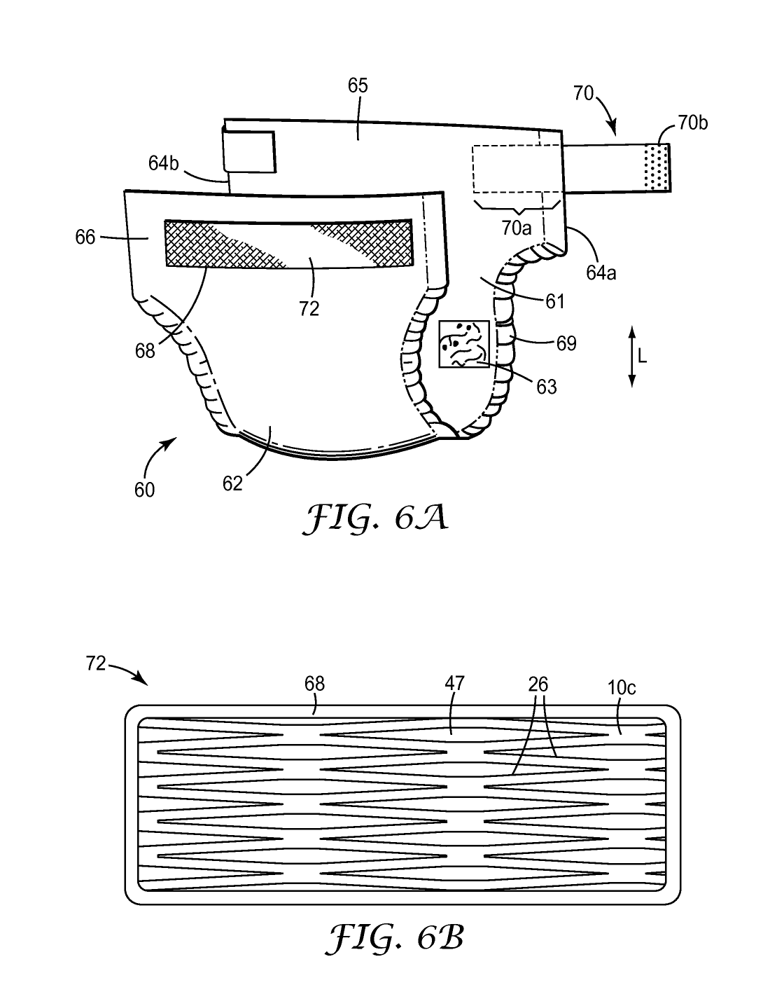

FIG. 6A is a schematic perspective view of one exemplary embodiment of an absorbent article according to the present disclosure. The absorbent article is a diaper 60 having an essentially hourglass shape. The diaper comprises an absorbent core 63 between a liquid permeable top sheet 61 that contacts the wearer's skin and an outwardly facing liquid impermeable back sheet 62. Diaper 60 has a rear waist region 65 having two fastening tabs 70 arranged at the two longitudinal edges 64a, 64b of diaper 60 and extending beyond longitudinal edges 64a, 64b of the diaper 60. The diaper 60 may comprise an elastic material 69 along at least a portion of longitudinal side edges 64a and 64b to provide leg cuffs. The longitudinal direction “L” of the absorbent article (e.g., diaper 60) refers to the direction that the article extends from the front to rear of the user. Therefore, the longitudinal direction refers to the length of the absorbent article between the rear waist region 65 and the front waist region 66. The lateral direction of the absorbent article (e.g., diaper 60) refers to the direction that the article extends from the left side to the right side (or vice versa) of the user (i.e., from longitudinal edge 64a to longitudinal edge 64b in the embodiment of FIG. 6A).

In FIG. 6A, fastening tabs 70 are secured through their manufacturer's end 70a to the rear waist region 65. The user's end 70b of the fastening tab 40 comprises any suitable hook strip. When attaching the diaper 60 to a wearer's body, the user's ends 70b of fastening tabs 70 can be attached to a target area 68 comprising the reticulated loop patch 72 according to the present disclosure, which may be arranged on the back sheet 62 of the front waist region 66.

An expanded view of the reticulated loop patch 72 is shown in FIG. 6B. The configuration of reticulated mechanical fastening strip 10c is shown and described above in FIG. 1C. However, the reticulated mechanical fastening strip may also be similar to that shown in any of FIGS. 1B, 2D, 3B, and 4B-4C. Adhesive 47 can be used to join the reticulated mechanical fastening strip to the back sheet 62. Exposed adhesive 47 may be present between at least some of the multiple strands 26 of reticulated mechanical fastening strip 10c to provide a combination of mechanical and adhesive fastening.

Although the embodiment illustrated in FIG. 6A is an absorbent article with attached fastening tabs, it is envisioned that the reticulated loop patch disclosed herein would be equally useful in absorbent articles with larger areas of hooks. For example, the ears of the absorbent article themselves comprise the loop material disclosed herein, or the absorbent article can have two target zones of loop material along the longitudinal edges of the back sheet in one waist region and two hook strips extending along the longitudinal edges of the absorbent article in the opposite waist region.

In use, fitting an absorbent article such as a diaper about the wearer usually requires the front and back waist portions of the diaper to overlap each other. As the diaper is worn the movements of the wearer tend to cause the overlapping front and back waist portions to shift position relative to each other. In other words, overlapping front and back waist portions are subjected to forces which tend to cause the front and back waist portions to assume a position relative to each other which is different from the position they assume when the diaper is initially fitted to the wearer. Such shifting can be made worse by the forces induced by the elastic at the leg openings. Unless such shifting is limited, the fit and containment characteristics of the diaper are degraded as the diaper is worn. The reticulated mechanical fastening strip according to and/or made according to the present disclosure may provide improved fit and closure stability by resisting such shifting. The resistance to shifting may be enhanced because relatively larger area and flexibility of the reticulated mechanical fastening strip disclosed herein.

The mechanical fastening strips according to and/or made according to the present disclosure may also be useful in many other fastening applications, for example, assembly of automotive parts or any other application in which releasable attachment may be desirable.

For any of the embodiments of the methods according to the present disclosure, interrupted slits in the backing can be formed, for example, using rotary die cutting of a continuous web of a loop material as described in any of the embodiments listed above. Interrupted slits can be made, for example, by using rotary cutting blades having gaps to form the bridging regions. The height of the blade in the gaps may be adjusted to allow for the bridging regions to be partially cut or not cut at all, depending on the desired embodiment. Other cutting methods (e.g., laser cutting) may also be used. Cutting can be performed from either surface of the continuous web.

In some embodiments of the methods of making a mechanical fastening strip according to the present disclosure, providing a slit loop material may be carried out by slitting through the backing in regions of the continuous web to provide interrupted slits while not slitting other regions. Typically, cross-web regions of interrupted slits made in the machine direction alternating with unslit regions may be made. The resulting continuous web may be rolled as a jumbo and stored until further processing. Alternatively, cutting through the unslit regions with a continuous cut (e.g., in the machine direction) can be carried out to provide separate webs of a slit loop material, which may be wound individually (e.g., level wound) into rolls and stored for later use.

When the mechanical fastening strip according to and/or made according to the present disclosure is a mechanical fastening patch cut to a desired size, interrupted slits may also be made in the loop material by hand, for example, using a razor blade.

For any of the methods of making a mechanical fastening strip according to the present disclosure, spreading the slit loop material to provide multiple strands of the loop material separated from each other between at least some of the bridging regions to provide at least one opening can be carried out in a variety of suitable ways. For example, spreading can be carried out on a continuous web using a flat film tenter apparatus, diverging rails, diverging disks, or a series of bowed rollers. When spreading is desired in the machine direction of a continuous web (e.g., with interrupted slits are made in the cross-web direction), monoaxial spreading in the machine direction can be performed by propelling the thermoplastic web over rolls of increasing speed, with the downweb roll speed faster than the upweb roll speed. When the mechanical fastening strip according to and/or made according to the present disclosure is a mechanical fastening patch cut to a desired size, spreading the slit loop material may also be carried out, for example, by hand.

In some embodiments, spreading the slit loop material may be carried out by passing the slit loop material over a smooth rounded element (e.g., half of a ball) with a diameter that is slightly smaller that the width of the slit loop material. Tension applied in the machine direction can cause the slits to open in the cross direction. Optionally, the rounded element may be heated.

The openings can be maintained between the multiple strands of the loop materials by joining the multiple strands to a carrier as described above. In other embodiments (e.g., embodiments with a significant extent of spreading), the openings are maintained by annealing the mechanical fastening strip. In some embodiments, annealing comprises heating the mechanical fastening strip. In some embodiments, annealing comprises heating and then cooling (e.g., rapidly cooling) the mechanical fastening strip to maintain its configuration. Heating may be carried out on a continuous web, for example, using heated rollers, IR irradiation, hot air treatment or by performing the spreading in a heat chamber.

In some embodiments, the loop material comprises a fibrous layer on a thermoplastic backing, and heating is only applied to the second surface of the thermoplastic backing (i.e., the surface opposite the fibrous layer). In embodiments where heated rollers are used, only rollers that are in contact with the thermoplastic backing are heated. When the mechanical fastening strip according to and/or made according to the present disclosure is a mechanical fastening patch cut to a desired size, heating the multiple strands of the backing may conveniently be carried out on a hot plate, for example.

In some cases, depending on the nature of the loop material and the extent of spreading, out-of-plane twisting can be observed when the slit loop material is spread. Such out-of-plane twisting can be controlled by maintaining or constraining at least some of the multiple strands in a substantially coplanar arrangement. A substantially “coplanar” arrangement refers to the strands occupying substantially the same plane. The term “substantially” in this regard can mean that at least some of the multiple strands can be twisted out of plane by up to 15, 10, or 5 degrees. “At least some” of the multiple strands being constrained refers to at least 25, 50, 75, or 90 percent or more of the multiple strands being constrained. In some embodiments, constraining at least some of the multiple strands is carried out while heating the multiple strands.

Maintaining at least some of the multiple strands in a substantially coplanar arrangement can be carried out, for example, by limiting the extent to which the slit loop material is spread. Providing the interrupted slits at an angle to the spreading direction (e.g., a 35 to 55 or 45 degree angle) may maintain at least some of the multiple strands in a substantially coplanar arrangement when the slit loop material is spread.

Constraining at least some of the multiple strands in a substantially coplanar arrangement can be carried out, for example, in a narrow gap that does not allow the strands to twist out of plane. In some embodiments, spreading the slit loop material is carried out in a narrow gap. In some embodiments, annealing the multiple strands is carried out within a narrow gap. The narrow gap can be formed in a variety of ways. When the mechanical fastening strip according to and/or made according to the present disclosure is a mechanical fastening patch cut to a desired size, the narrow gap can be formed between a hot plate and a cold plate. When the loop material comprises a fibrous layer on a backing, the backing can be placed on the hot plate, and a cold plate can be held against the fibrous layer with light pressure to press the multiple strands into a substantially coplanar arrangement. Typically, the slit loop material can be spread incrementally, pressed between a hot plate and a cold plate, and allowed to cool to maintain the openings between the multiple strands and to constrain the multiple strands in a substantially coplanar arrangement. The process can be repeated until the desired amount of spreading is reached.

Constraining a mechanical fastening strip in a continuous web process can be carried out with a narrow gap between hot and cold surfaces used in connection with the diverging disks or other spreading apparatus described above. It is possible in a continuous web process to incrementally spread the slit loop material, for example, with a series of bowed rollers, and anneal by heating and cooling with alternating heated and cooled rollers. In the embodiments in which a slit loop material is passed over a smooth rounded element, the tension in the machine direction may limit out-of-plane twisting.

In some embodiments where the loop material comprises a fibrous layer and a thermoplastic backing, the thermoplastic backing can be joined to a fibrous web carrier. In some of these embodiments, the joining comprises impinging heated gaseous fluid (e.g., ambient air, dehumidified air, nitrogen, an inert gas, or other gas mixture) onto a first surface of the fibrous web carrier while it is moving; impinging heated fluid onto the second surface of the thermoplastic backing while the continuous web is moving, wherein the second surface is opposite the fibrous layer on the backing; and contacting the first surface of the fibrous web carrier with the second surface of the backing so that the first surface of the fibrous web is melt-bonded (e.g., surface-bonded or bonded with a loft-retaining bond) to the second surface of the backing. Impinging heated gaseous fluid onto the first surface of the fibrous web and impinging heated gaseous fluid on the second surface of the backing may be carried out sequentially or simultaneously.

Further methods and apparatus for joining a continuous web of loop material with a thermoplastic backing to a fibrous carrier web using heated gaseous fluid may be found in U.S. Pat. Appl. Pub. Nos. 2011/0151171 (Biegler et al.) and 2011/0147475 (Biegler et al.), incorporated herein by reference in their entirety.

This disclosure may take on various modifications and alterations without departing from its spirit and scope. Accordingly, this disclosure is not limited to the above-described embodiments but is to be controlled by the limitations set forth in the following claims and any equivalents thereof. This disclosure may be suitably practiced in the absence of any element not specifically disclosed herein. All patents and patent applications cited above are hereby incorporated by reference into this document in their entirety.

What is claimed is:

1. A method of making a mechanical fastener, the method comprising:
slitting through a loop material to provide a slit loop material having interrupted slits, wherein each interrupted slit is interrupted by at least one intact bridging region of the slit loop material;
spreading the slit loop material to provide multiple strands of the loop material attached to each other at least at some of the bridging regions and separated from each other between at least some of the bridging regions to provide at least one opening; and
fixing the multiple strands of the loop material in a spread configuration to maintain the at least one opening between the multiple strands.

2. The method of claim 1, wherein the loop material comprises a fibrous layer disposed on a backing.

3. The method of claim 2, wherein the backing is a thermoplastic backing, and wherein fixing the multiple strands comprises annealing.

4. The method of claim 2, further comprising maintaining at least some of the multiple strands in a substantially coplanar arrangement.

5. The method claim 2, wherein the fibrous layer comprises a nonwoven material.

6. The method of claim 2, wherein the fibrous layer comprises arcuate portions projecting in the same direction from spaced anchor portions on the backing.

7. The method of claim 2, further comprising joining the multiple strands of the loop material to a fibrous carrier, wherein the backing is a thermoplastic backing, and wherein a surface of the thermoplastic backing opposite the fibrous layer is surface-bonded to the fibrous carrier.

8. The method of claim 1, wherein the loop material is a web of indefinite length having a machine direction, and wherein the interrupted slits extend in the machine direction.

9. The method of claim 1, further comprising joining the multiple strands of the loop material to a carrier by applying adhesive to at least one of the carrier, the loop material before it is slit, or the multiple strands of the loop material.

10. The method of claim 1, further comprising joining the multiple strands of the loop material to a carrier, wherein at least the portion of carrier to the multiple strands are joined has up to a ten percent elongation in a direction perpendicular to the direction of the interrupted slits.

11. The method of claim 1, wherein at least one of the following conditions is met:
for any two adjacent interrupted slits, the bridging regions are staggered in a direction transverse to the interrupted slits; or
wherein the interrupted slits are substantially evenly spaced apart from each other.

12. The method of claim 1, wherein the interrupted slits cut through the entire thickness of the loop material.

13. The method of claim 1, wherein the interrupted slits are partial depth slits that allow the loop material to open during the spreading.

14. A reticulated mechanical fastening laminate comprising a loop material having a regular pattern of spaced apart geometric shaped openings joined to a carrier, wherein at least the portion of carrier to which the loop material is joined has up to a ten percent elongation.

15. The reticulated mechanical fastening laminate of claim 14, further comprising adhesive between at least a portion of the loop material and at least a portion of the carrier, wherein the adhesive is optionally exposed in the geometric shaped openings, and wherein the loop material and the carrier are optionally different colors.

16. A reticulated mechanical fastening web comprising a loop material having a regular pattern of spaced apart geometric shaped openings, wherein the loop material is not joined to an elastic or pleated extensible carrier.

17. The reticulated mechanical fastening web of claim 16, wherein the loop material comprises multiple strands of the loop material attached to each other at bridging regions in the loop material and separated from each other between the bridging regions to provide the geometric shaped openings.

18. The reticulated mechanical fastening web of claim 16, wherein the loop material comprises a fibrous layer disposed on a backing.

19. The reticulated mechanical fastening web of claim 18, wherein the fibrous layer comprises arcuate portions projecting in the same direction from spaced anchor portions on the backing.

20. An absorbent article comprising the reticulated mechanical fastening web of claim 16.
